(19) United States
(12) Patent Application Publication
(10) Pub. No.: US 2014/0379038 A1
(43) Pub. Date: Dec. 25, 2014

(54) SYSTEM FOR ANATOMICAL REDUCTION OF BONE FRACTURES

(71) Applicants: Sanja Dogramadzi, Bristol (GB); Roger Michael Atkins, Bristol (GB); Daniel Raabe, Bautzen (DE)

(72) Inventors: Sanja Dogramadzi, Bristol (GB); Roger Michael Atkins, Bristol (GB); Daniel Raabe, Bautzen (DE)

(73) Assignee: UNIVERSITY OF THE WEST OF ENGLAND, BRISTOL, Bristol (GB)

(21) Appl. No.: 14/343,583

(22) PCT Filed: Sep. 7, 2012
(86) PCT No.: PCT/GB2012/000703

§ 371 Date: Jul. 23, 2014

(30) Foreign Application Priority Data
Sep. 9, 2011 (GB) 1115586.8

(51) Int. Cl. A61B 17/88; A61B 19/00

(52) U.S. Cl. CPC A61B 17/8866; A61B 19/50; A61B 19/22; A61B 19/56; A61B 19/2203; A61B 2019/564
USPC 606/86 R

(57) ABSTRACT

The present application relates to a system (10) for anatomical reduction of bone fractures in which first and second manipulators (12, 14), and optionally a third manipulator (16), are attached to fragments of the fracture to be reduced by percutaneous attachment devices such as Schanz pins. An underlying processing system determines, from one or more medical images of the fracture, manipulations such as rotations and translations of the bone fragments required to correctly reposition and align the fragments for optimum healing of the fracture. The processing system provides motion reference signals (position, speed, acceleration and force) for a controller, which in turn causes the first, second and third manipulators (12, 14, 16) to effect the calculated manipulations.

### TECHNICAL FIELD

The present application relates to a system for anatomical reduction of bone fractures.

### BACKGROUND OF THE INVENTION

For optimum healing of bone fractures in the human body, to ensure that the bone and surrounding joints are able to function correctly again, the fragments of the broken bone must be subjected to an anatomical reduction, which involves positioning and aligning the fragments of the broken bone to reconstruct the fractured bone as precisely as possible, so that the bone recovers to a form as close as possible to its original form as it heals.

This anatomical reduction may be performed by open surgery, in which large incisions are made in flesh around the affected joint and the bone fragments are manipulated by a surgeon to reposition and realign them as precisely as possible. Whilst this technique can be effective, it has disadvantages. For example, the anatomical reduction is not always perfect, in that the bone fragments are not always perfectly positioned or aligned. This leads to imperfect healing and can cause arthritis later in the patient's life. Additionally, the extensive exposure required by the open surgery procedure typically slows bone healing and produces unsightly scars, as well as giving rise to an increased risk of infection. A prolonged period of post-operative rehabilitation is required, which requires the patient to endure an extended stay in hospital.

In order to mitigate the disadvantages of open surgery techniques, minimally invasive percutaneous procedures have been developed. These techniques involve sequentially fixating and manipulating each bone fragment manually, without making large incisions in the patient's flesh. Such techniques are associated with a faster recovery and a lower risk of infection compared to open surgery techniques. However, minimally invasive techniques may involve lower reduction accuracy, and in some cases the reduction accuracy is less than the minimum accuracy (typically <1 mm translationally and <5 degrees rotationally) required for optimum clinical outcomes. Moreover, minimally invasive procedures require multiple radiographic images of the patient to be taken during the surgical procedure to ensure that the bone fragments are being correctly positioned and orientated during the procedure. This exposes the patient and medical staff to undesirably high levels of radiation. Notwithstanding these multiple radiographic images, fragment reduction remains sub-optimal.

Accordingly, a need exists for a system which improves the level of reduction accuracy of minimally invasive surgical techniques and reduces exposure to radiation, whilst retaining the recovery speed and low infection risk of existing minimally invasive techniques.

### SUMMARY OF INVENTION

The present application relates to a system for anatomical reduction of bone fractures in which first and second manipulators, and optionally a third manipulator, are attached to fragments of the fracture to be reduced by percutaneous attachment devices such as Schanz pins. An underlying processing system determines, from one or more medical images of the fracture, manipulations such as rotations and translations of the bone fragments required to correctly reposition and align the fragments for optimum healing of the fracture. The processing system provides motion reference signals (position, speed, acceleration and force) for a controller, which in turn causes the first, second and third manipulators to effect the calculated manipulations.

According to a first aspect of the present invention there is provided a system for reduction of bone fractures, the system comprising: a first manipulator for manipulating a bone section of the fracture, the first manipulator being attachable to the bone section by means of a percutaneous attachment device; a second manipulator for manipulating a first bone fragment of the fracture, the second manipulator being attachable to the first fragment by means of a percutaneous attachment device; and a processing system configured to determine reference signals for the first and second manipulators required to effect manipulations of the bone section and the first fragment required for correct anatomical reduction of the fracture; and a controller configured to control the first and second manipulators to cause them to perform the manipulations of the bone section and the first bone fragment.

The system of the first aspect of the present invention permits minimally invasive semi-automated anatomical reduction of intra-articular joint and other fractures such as long bone fractures. By determining manipulations required for correct reduction of the fragment using processing means, and implementing the manipulations using manipulators controlled by control means greater reduction accuracy can be achieved than with conventional manually performed minimally invasive surgical techniques.

Preferably, the first manipulator is a parallel manipulator comprising first and second end sections connected by a plurality of linear actuators.

The system may further comprise a third manipulator for manipulating a second bone fragment of the fracture, the third manipulator being attachable to the fragment by means of a percutaneous attachment device, wherein the processing system is configured to determine reference signals for the third manipulator required to effect manipulations of the second fragment required for correct anatomical reduction of the fracture and the controller is further configured to control the third manipulator to perform the manipulations of the second bone fragment.

The system according may further comprise a fourth manipulator having a tool for removing fragments of bone that cannot be manipulated.

Preferably, the second and/or third and/or fourth manipulator is a parallel manipulator comprising a fixed base and a moveable platform connected to the fixed base by a plurality of linear actuators.

For example, the fixed base may be connected to the moveable platform by six linear actuators.

The controller may be configured to cause the manipulators to perform the manipulations of the reference bone and the bone fragment(s) substantially simultaneously.

The processing system may be configured to: receive an image of the fracture; segment the image of the fracture to identify fracture surfaces of the reference bone and the bone fragment(s) of the fracture; generate a fragment surface layer for the reference bone and the fragment(s) representative of surfaces of the reference bone and the bone fragments; display a graphical representation of the reference bone and the bone fragments; receive a user input; manipulate the graphical representation to simulate reduction of the fracture based on the user input received; record the manipulations of the graphical representation; and, based on the recorded manipulations, determine the reference signals for the manipulations required for correct anatomical reduction of the fractured bone.

The user input may be received by means of a virtual joystick presented as part of a graphical user interface by the processing system.

According to a second aspect of the invention there is provided a method for calculating manipulations required to effect an anatomical reduction of a bone fracture, the method comprising the steps of: segmenting an image of the fracture to identify fracture surfaces of bone fragments of the fracture; generating a fragment surface layer for each fragment representative of surfaces of the bone fragment; calculating an axis of each bone fragment; calculating fracture surfaces for each bone fragment; and calculating, based on the axes and fracture surfaces calculated, manipulations required for reduction of the fracture.

The segmenting of the image of the fracture may comprise calculating Hounsfield intensity values for structures shown in the image and identifying the fracture surfaces from the Hounsfield intensity values.

Generating the fragment surface layer for each fragment may comprise using data from the segmented image to generate a point cloud representing each bone fragment and performing a triangulation on the point cloud.

Calculating the axis of a bone fragment may comprise defining two sections of the bone fragment, calculating the point centre of each of the defined sections of the fragment and calculating an axis vector for the bone fragment by subtracting the point centre of one section from the point centre of the other section.

Calculating the point centre of a section of the bone fragment may comprise calculating the mean x, y and z coordinates for each point of the point cloud in the section.

Calculating the manipulations required for reduction of the fracture may comprise calculating a manipulation required for axial alignment of the fragments, calculating an angle of rotation required for fracture surface alignment and calculating a translation required to close a gap between the bone fragments.

Calculating the manipulation required for axial alignment of the fragments may comprise calculating a transformation required to align the axis vectors of the fragments.

Calculating the angle of rotation required for fracture surface alignment may comprise generating a first polyline representative of the fracture surface of a first, reference fragment; generating a plurality of second polylines representative of the fracture surface of a second fragment, each second polyline having an incremental angular offset with respect to the first polyline; performing a comparison of the first and each of the plurality of second polylines, wherein the angle of rotation required is determined by calculating the angular offset for which the comparison determines that the first and second polylines are most similar.

Performing the comparison may comprise: calculating a first cross correlation coefficient for the first polyline; for each of the plurality of second polylines, calculating a second cross correlation coefficient; and calculating a difference value between the first and second cross correlation coefficients, wherein the angle of rotation required is the angular offset of the second polyline for which the difference value is smallest.

According to a third aspect of the invention there is provided a method for calculating manipulations required to effect an anatomical reduction of a bone fracture, the method comprising the steps of: segmenting an image of the fracture to identify fracture surfaces of bone fragments of the fracture; generating a fragment surface layer for each fragment representative of surfaces of the bone fragment; displaying a graphical representation of the bone fragments; receiving a user input; manipulating the graphical representation of one of the bone fragments to simulate reduction of the fracture based on the user input received; recording the manipulations of the graphical representation of the bone fragment; and, based on the recorded manipulations, determining reference signals for manipulations of the fragments of fractured bone required for correct anatomical reduction of the fractured bone.

The user input may be received by means of a virtual joystick presented as part of a graphical user interface.

The processing means of the system of the first aspect may be configured to perform the method of the second or third aspects, and the controller may be configured to cause the manipulators to perform the calculated manipulations.

According to a fourth aspect of the invention there is provided a computer program which, when executed on appropriate processing means, performs the method of the second or third aspect.

### BRIEF DESCRIPTION OF THE DRAWINGS

Embodiments of the invention will now be described, strictly by way of example only, with reference to the accompanying drawings, of which:

FIG. 1 is a schematic perspective representation of a part of a system for anatomical reduction of bone fractures;

FIG. 2 is an alternative view of the system illustrated in FIG. 1;

FIG. 3 is a schematic representation of a first manipulator used in the of the system illustrated in FIGS. 1 and 2;

FIG. 4 is a schematic representation of a second and third manipulator used in the system illustrated in FIGS. 1 and 2;

FIG. 5 is a flow diagram illustrating steps taken prior to and during a surgical procedure to reduce a part distal femoral fracture performed using the system of FIGS. 1 to 4;

FIG. 6 is a schematic representation of a distal femoral bone fracture;

FIG. 7 is a CT image showing a horizontal view of a bone fracture that has been processed to show Hounsfield intensities of different portions of the fracture;

FIG. 8 is a point cloud representing a two-part long bone fracture (diaphyseal fracture);

FIGS. 9 and 10 illustrate steps taken in determining an axis of a bone fragment as part of a process for reducing the fracture;

FIG. 11 illustrates a graphical representation of a two part diaphyseal fracture for which the axes of the bone fragments have been aligned;

FIG. 12 illustrates the result of a step of angular alignment of the bone fragments illustrated in FIGS. 8 to 11;

FIG. 13 is a schematic representation of the second manipulator illustrated in FIG. 4;

FIG. 14 is a flow chart illustrating steps performed as part of a process to determine a reference signals of linear actuators of the manipulators illustrated in FIGS. 1 and 2 to achieve a desired and defined manipulation of a bone fragment; and

FIG. 15 is a flow chart illustrating steps performed to calculate Forward Kinematics of the manipulators illustrated in FIGS. 1 and 2.

FIGS. 16 to 18 are screenshots of a graphical user interface used in an alternative embodiment of a system for anatomical reduction of fractures.

### DESCRIPTION OF THE EMBODIMENTS

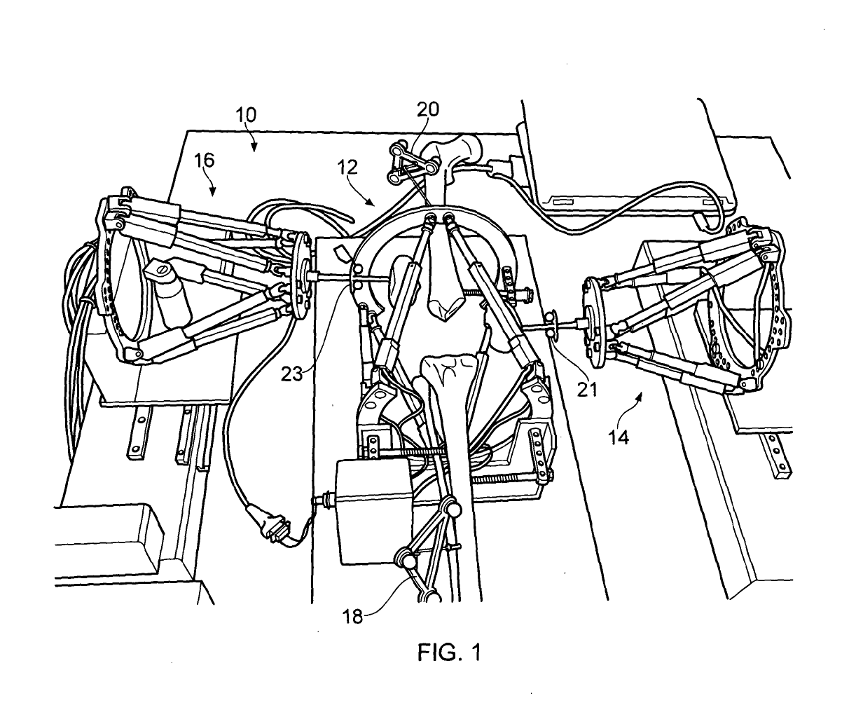

FIGS. 1 to 3 are schematic illustrations of part of a system for anatomical reduction of bone fractures. The system, illustrated generally at 10, includes a first manipulator 12 for restoring limb length and alignment and for decompressing the fracture, a second manipulator 14 for manipulating a first fragment of a fractured bone and a third manipulator 16 for manipulating a second fragment of fractured bone. In the case of two-part fractures the third manipulator 16 may not be required.

The system 10 also employs a motion recording system which uses up to eight cameras (not shown) to register motion of bone fragments. The motion recording system uses a reference marker frame 20 carrying retro-reflective markers that is rigidly attached to a selected reference bone of the patient, and an active marker frame 18 carrying retro-reflective markers rigidly attached to a bone fragment that is to be aligned as part of the reduction of the fracture. Additional active marker frames 21, 23 carrying retro-reflective markers are rigidly attached to the first and second bone fragments that are to be manipulated (e.g. rotated and translated) as part of the reduction of the fracture. The reference marker frame 20 and the active marker frames 18, 21, 23 can be detected by the camera(s) of the motion recording system, with the reference marker frame 20 providing a reference or origin against which movement of the active marker frames 18, 21, 23 (and therefore movement of the bone fragments to be reduced) can be recorded. The motion recording system is able to record movement of the active marker frames 18, 21, 23, and therefore of the bone fragments to which the active marker frames 18, 21, 23 are attached, in six degrees of freedom.

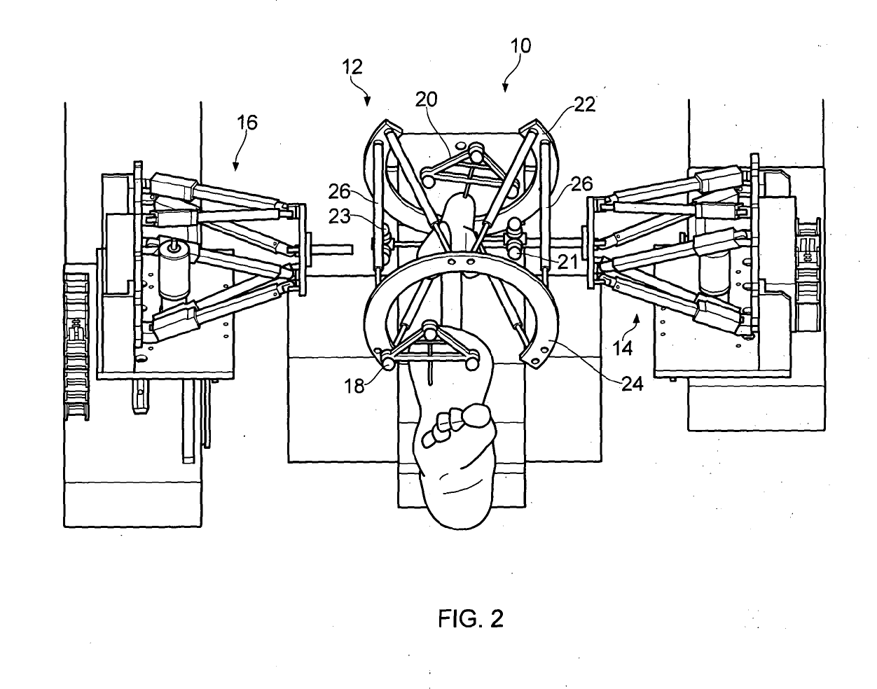

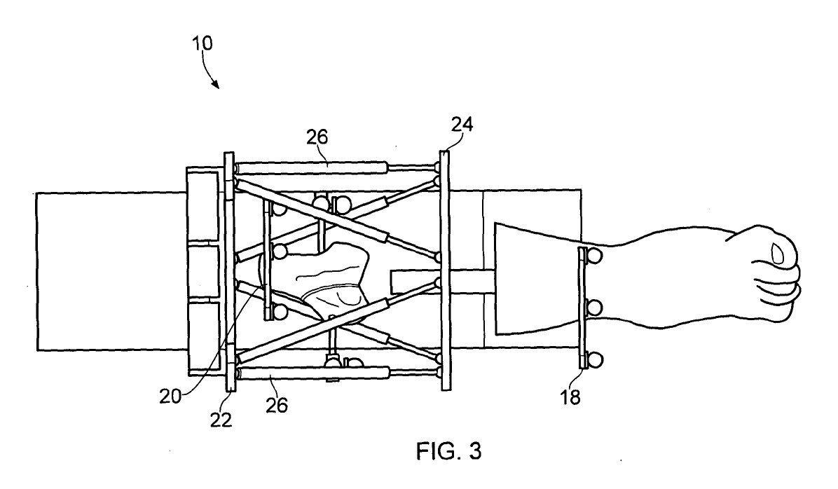

FIGS. 2 and 3 are alternative views of the system. 10 illustrated in FIG. 1, from which it can be more clearly seen that the first manipulator. 12 in this example is a lightweight parallel manipulator in the form of a Gough Stewart platform made up of first and second parallel, partially annular, end sections 22, 24 connected by a number (in this example 6) of linear actuators 26, such that the first manipulator 12 is able to correct in six degrees of freedom through the linear actuators 26. The first end section 22 of the first manipulator 12 is fixed such that changes in the length of the linear actuators 26 cause the second end section 24 to move relative to the first end section 22. As is explained below, the first manipulator 12 remains in place during a surgical procedure for reducing a fracture. The procedure requires high resolution medical images such as CT images to be taken, and thus the first manipulator should be constructed of a material that does not impede the taking of such images.

The first manipulator 12, which is also referred to as an “external robot”, is configured to extend and align long bones around the fracture that is to be reduced. To this end, the first manipulator 12 can be connected to the long bones around the fracture site by means of pins and wires (not shown), each having a first end which is screwed into or inserted in a hole drilled in the long bone by a surgeon, and a second end that is attached to the first end section 22. The length of the linear actuators 26 of the first manipulator 12 can then be adjusted to achieve the desired spacing and angulation between the first and second end sections 22, 24 of the of the first manipulator 12, which in turn extends and aligns the long bones around the fracture. In the example illustrated in FIG. 2 the first manipulator 12 is used to align the tibia with respect to the femur.

By extending and aligning long bones using the first manipulator 12 in this way, a workspace is created around the fracture in which the second manipulator 14 (and the third manipulator 16, if appropriate) can operate to rotate and translate a bone fragment, as is described in more detail below. As the first manipulator 12 remains attached to the patient's bones following the surgical procedure, it assists in maintaining the correct position and orientation of bone fragments during post-operative healing of the fracture.

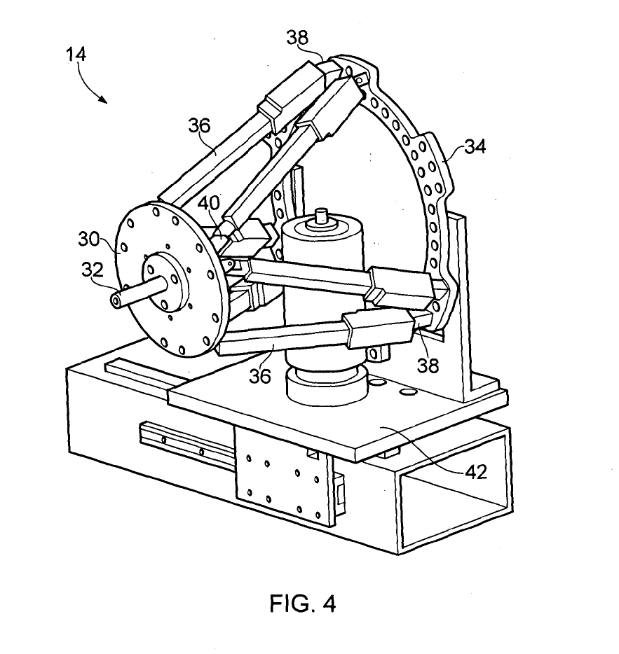

FIG. 4 is a schematic illustration showing the second manipulator 14 in more detail. The third manipulator 16 is similar in construction and operation to the second manipulator 14. The second manipulator 14, which is also referred to as an “internal robot”, a lightweight parallel manipulator, and is configured to manipulate a fragment of bone in and around the fracture site by effecting rotational and translational movement of the bone fragment so as to achieve the correct position and orientation for a successful reduction of the fracture, that is to say reconstruction of the fractured bone with the fragments in the correct position and orientation.

The second manipulator 14 and the third manipulator 16 each take the form of a hexapod robot, having a platform 30 on which an end effecter 32 is mounted. The platform 30 is connected to a fixed partially annular base 34 by means of six linear actuators 36 (hence the term “hexapod robot”). Each linear actuator 36 is connected at one end to the base 34 by means of a f=2 universal joint 38 and at the other end to the platform 30 by means of a f=2+1 universal joint 40. This arrangement permits precise movement of the end effecter 32 with up to six degrees of freedom. The end effecter 32 itself is mounted for rotation about its longitudinal axis, whilst in the example illustrated in FIG. 4, the base 34 is attached to a linearly moveable table 42 to increase the workspace of the second manipulator 14 and to permit linear movement of the second manipulator 14 in a plane generally parallel to the base 34.

In an alternative embodiment, a generally semi-circular track may be provided for mounting the second and third manipulators 14, 16. The semi-circular track is in the form of an arch which extends upwardly from a generally circular base that can be rotated through 360 degrees. In this embodiment, the fracture to be treated is positioned within the arch, above the base, and the second and third manipulators 14, 16 are mounted on the arch by means of adjustable articulated attachment devices, so that each of the second and third manipulators 14, 16 can be fixed in position with respect to the centre of mass of the fragment to be manipulated by the respective robot. The rotatable base and arched track arrangement defines a generally hemispherical envelope in which the second and third manipulators can operated, permitting good accessibility by the second and third manipulators to the fracture to be treated.

The linear actuators 36, the end effecter 32 and the linearly moveable table 42 (or rotatable base) are electrically operable components which are connected to a controller (not shown) which controls the movements of the second manipulator 14 to achieve highly precise and accurate rotation and translation of a bone fragment to which the end effecter 32 is attached by means of a pin such as a Schanz pin inserted into the bone fragment.

Similarly, the linear actuators 26 of the first manipulator 12 are also electrically operable, and are connected to the controller, so as to permit precise control of the degree of extension of each strut 26, and therefore the spacing and angular displacement between the first and second end sections 22, 24 of the of the first manipulator 12 to achieve a desired alignment and spacing of the long bones around the fracture site.

The exemplary system 10 illustrated in FIGS. 1 to 3 includes a first manipulator 12, also referred to as an external robot, and second and third manipulators 14, 16, also referred to as internal robots. It will be appreciated that additional manipulators of the kind illustrated in FIG. 4 may also be provided as further internal robots, mounted either on moveable tables, or on the generally semi-circular arched track discussed above. For example, a fourth manipulator may be provided to extend the functionality of the system 10.

Such a further manipulator may be provided with a tool for extracting small fragments of bone that cannot be manipulated from the fracture site, under control of the controller.

Additionally or alternatively, one or more further manipulators may be provided, again mounted either on moveable tables or on the arched track discussed above, having a camera or other imaging system, or other instruments such as physical or radiography probes that can be inserted into the fracture site under the control of the controller to permit visualisation of the fracture and/or collection of data relating to particular parameters of the fracture such as distances, angles, fracture surfaces and the like.

Similarly, one or more further manipulators may be provided, again mounted either on moveable tables or on the arched track discussed above, and used to place and secure attachment devices such as plates, nails, screws or any other suitable attachment devices to the bone fragments following reduction of the fracture, to stabilise the fracture once it has been reduced.

The controller receives signals from an underlying processor or processing system such as a general purpose computer running appropriate software (not shown) which calculates the rotations and translations required to position and align (i.e. reduce) the bone fragment manipulated by the second manipulator 14 (and the bone fragment manipulated by the third manipulator 16, where provided) correctly for optimum healing, based on images, such as CT (computer tomography) scans, of the fracture and the surrounding tissue taken before and during the reduction procedure. The processing system also calculates reference signals required by the manipulators 14, 16 to effect the manipulations calculated. Each manipulator 14, 16 has a feed drive controller which is configured to minimise the error between the reference input signals received from the controller of the processing system and positional feedback signals received from an absolute displacement transducer associated with each actuator 36.

The processing system also calculates the alignment and extension of the long bones around the fracture site required to restore limb length and limb alignment for optimum healing of the fracture, again based on medical images taken of the fracture and the surrounding tissue before and during the reduction procedure, and Cartesian motion feedback from the motion recording system. The processing system also calculates and transmits reference signals indicative of the required alignment and extension of the long bones to a position controller of the first manipulator 12 to cause it to adjust the length of one or more of the linear actuators 26 so as to effect the calculated alignment and extension of the long bones around the fracture site, as will be described in more detail below. The first, second and third manipulators 12, 14, 16 may be controlled simultaneously to effect their respective manipulations on the fracture in parallel.

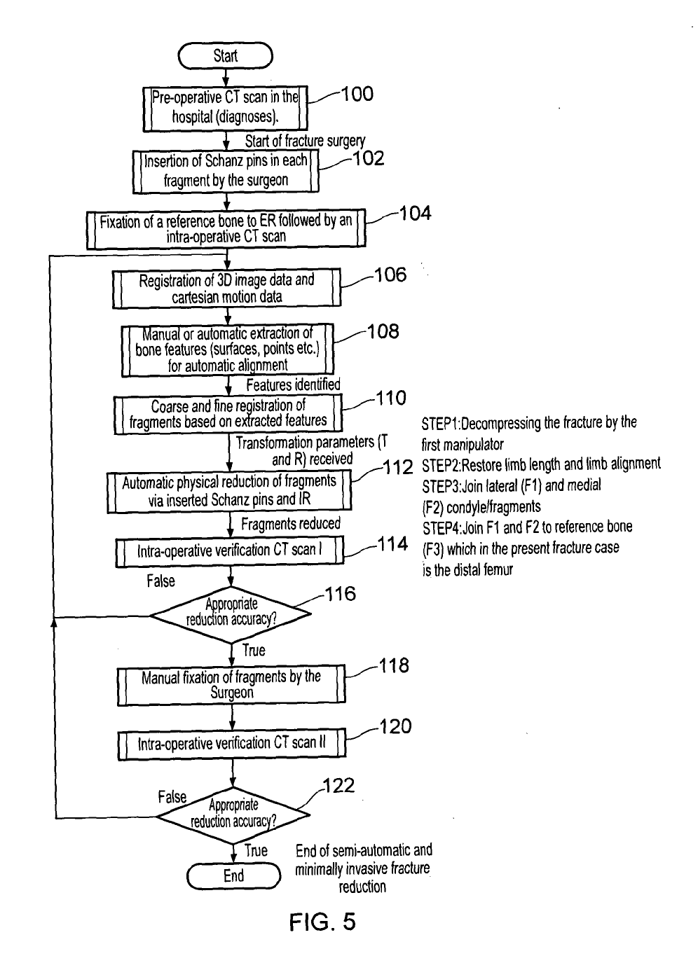

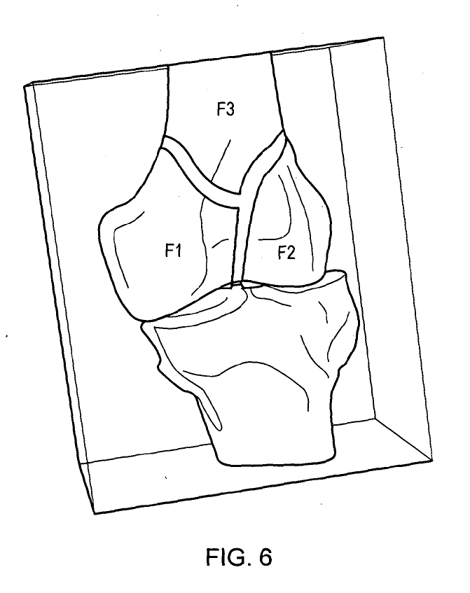

FIG. 5 is a flow diagram illustrating steps taken prior to and during a surgical procedure performed using the system of FIGS. 1 to 4. In this example the system is used to perform a minimally invasive surgical reduction of a three part intra-articular distal femoral fracture of the type illustrated in FIG. 6.

In a first step 100 a CT scan is taken of a suspected fracture and surrounding tissue for the purpose of diagnosing the patient's injury. This CT scan enables a surgeon to identify fragments (shown as F1 and F2 in FIG. 6) of a fractured bone. Additionally, data from this pre-operative CT scan enables a medical engineer to digitally segment the fracture in a preliminary step, to assist in the identification of bone fragments.

In the operating theatre, Schanz pins are inserted in the identified bone fragments by the surgeon (step 102), by means of which the bone fragments can be manipulated by the second manipulator 14 and, if appropriate, the third manipulator 16. In step 104 a reference bone (in this example the femur, shown as F3 in FIG. 6) is fixated (i.e. attached) to the first manipulator 12 by means of Schanz pins inserted into the reference bone and attached to the first end section 22 of the first manipulator 12. The reference bone F3, to which the marker frame 20 is rigidly attached by means of a pin, will be used as a reference point for the assembly of the two other fragments F1 and F2. A further, inter-operative, CT scan of the fracture site is then taken to ensure that the locations and dimensions of the fragments F1 and F2 have not changed with respect to the previous scan.

At step 106, 3D image data from a further CT scan and from the motion recording system are registered by the processing system. This step is required to ensure that both data sets are referenced to the same coordinate frames, to ensure that rotations and translations of bone fragments performed during the procedure have the expected effect.

In step 108 features such as surfaces, points, contours and the like of the bone fragments present in the image are identified and extracted for use in a later matching step in which pairs of bone fragment which match, i.e. belong together, are identified and the appropriate rotations and translations of the matching bone fragment pairs to achieve the optimum reduction of the fracture are determined. The matching step is not required where there are only two bone fragments, as the two fragments will clearly belong to a single pair, but for multiple fragment fracture cases matching is required.

The features may be identified and extracted automatically, or may be identified by a qualified orthopaedic surgeon using a user interface which presents the images of the bone fragments and permits the surgeon to manipulate the images of the bone fragments, or to select matching features of the bone fragments, or both, for use in determining the required rotations and translations.

At step 110 the identified features of the bone fragments are used by the processing system in an automatic registration process which calculates the required translations and rotations of the bone fragments to achieve the desired optimum reduction of the fracture. This automatic registration process is described in more detail below.

Once the required translations and rotations of the bone fragments have been calculated they are passed, at step 112, to the controller. The controller causes the first manipulator 12 to create a workspace in which the second manipulator 14 (and if appropriate the third manipulator 16) are able to work, by decompressing the fracture by lateral extension of the reference bone. The processing system then translates the translations and rotations into control signals that are used by the controller to control the second manipulator 14 (and, if appropriate, the third manipulator 16) to cause the end effecter 32 to effect the required translations and rotations of the bone fragment to which it is attached. The controller also causes the first manipulator 12 to restore the active bone (i.e. the bone to which active marker frame 18 is attached) to its correct position and orientation with respect to the reference bone F3.

Once the translations and rotations have been effected by the second manipulator 14 and the active bone (in this example the tibia) has been restored to its correct position and orientation by the first manipulator 12 a further CT scan is taken, at step 114, to verify that the reduction of the fracture has been achieved to the desired level of accuracy. If the reduction has not been achieved to a satisfactory level of accuracy steps 106 to 114 are repeated. If the reduction has been achieved to a satisfactory level of accuracy the bone fragments are fixated manually by the surgeon with respect to the first manipulator 12 at step 118 and a further CT scan is taken at step 120, to ensure that the fragments have not been displaced during or after the manual fixation step.

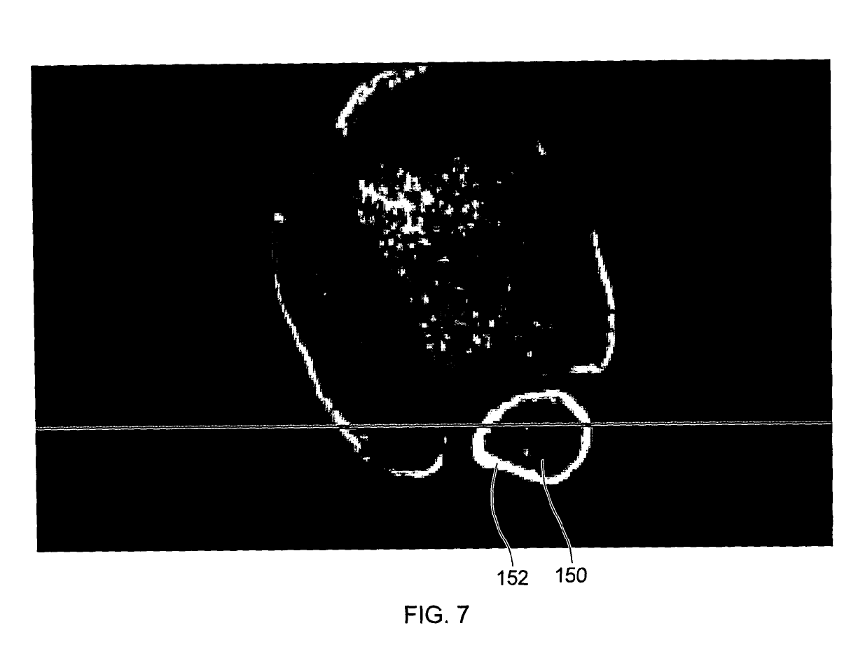

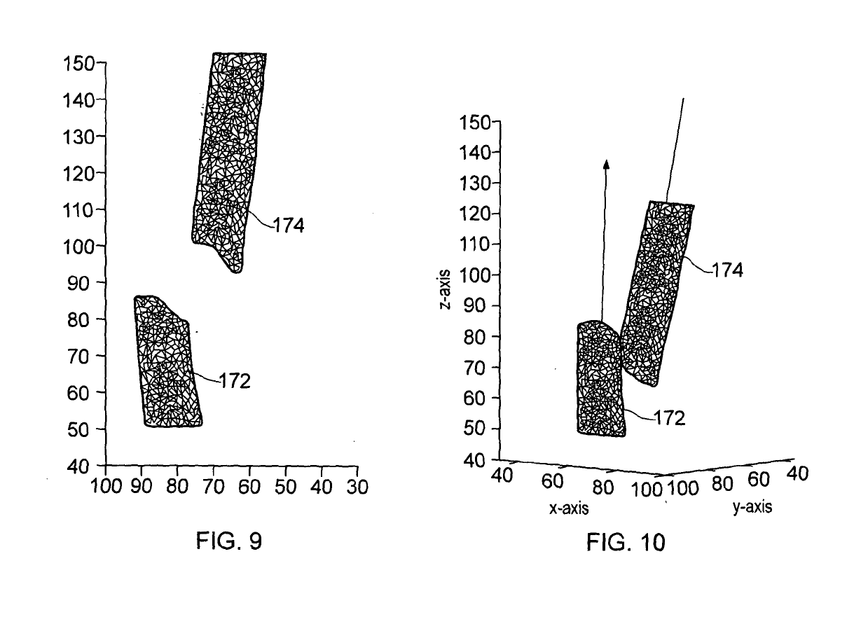

The process used by the system 10 for reduction of a bone fracture, used in steps 106 to 110 of the surgical procedure illustrated in FIG. 5 will be now described in more detail with reference to FIGS. 7 to 12.

As is discussed above, a first part of the reduction process involves the segmentation of bone from soft tissue in the image produced by the pre-operative CT scan. This segmentation is carried out by the processing system, which calculates Hounsfield intensities of the structures shown in the images produced by the CT scan. As is well known, different structures in the human body have different Hounsfield intensities when imaged by a CT scan. For example, the inner part of bone, shown at 150 in FIG. 7, has a lower density than the surface part of bone 152. This property can be used by the processing system to identify and segment fracture surfaces from other sections of the bone, since fracture surfaces have Hounsfield intensities similar to those of the inner section of the bone.

Once the bone fragments have been identified by the segmentation step, a matching step may be required to match a fracture surface of one bone fragment with a corresponding fracture surface of another bone fragment. This matching step may not be required for fractures where there are only two fragments, such as simple long bone fractures, since the fracture surfaces of the two fragments must match. However, for more complex fractures having more than two fragments matching using extracted bone features is typically required.

This matching step may be performed by a qualified orthopaedic surgeon using a user interface which presents the images of the bone fragments and permits the surgeon to identify the matching fracture surfaces of different bone fragments based on features such as contours, points or extracted and calculated surface areas in mm². Alternatively, the processing system may automatically identify the features automatically, and may use these features to identify matched pairs of fracture surfaces.

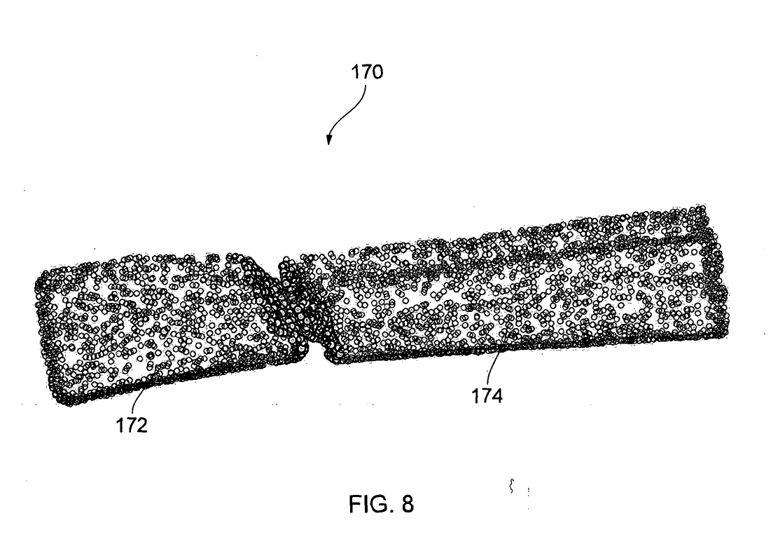

Once the CT images have been processed to identify and segment the bone fragments, a point cloud of the segmented 3D bone shape is generated, based upon data from the segmented images of the fracture, for each bone fragment. Each point cloud is a matrix of Cartesian coordinates of points representing the surface of the bone fragments, and can be represented graphically, as is shown at 170 in FIG. 8. It will be noted that the point cloud illustrated in FIG. 8 represents a long bone fracture (in this instance a diaphyseal fracture), but it will be appreciated that the techniques described herein are equally applicable to other fracture cases, for example restoration of bone length and alignment of the two part femoral fracture illustrated in FIG. 6.

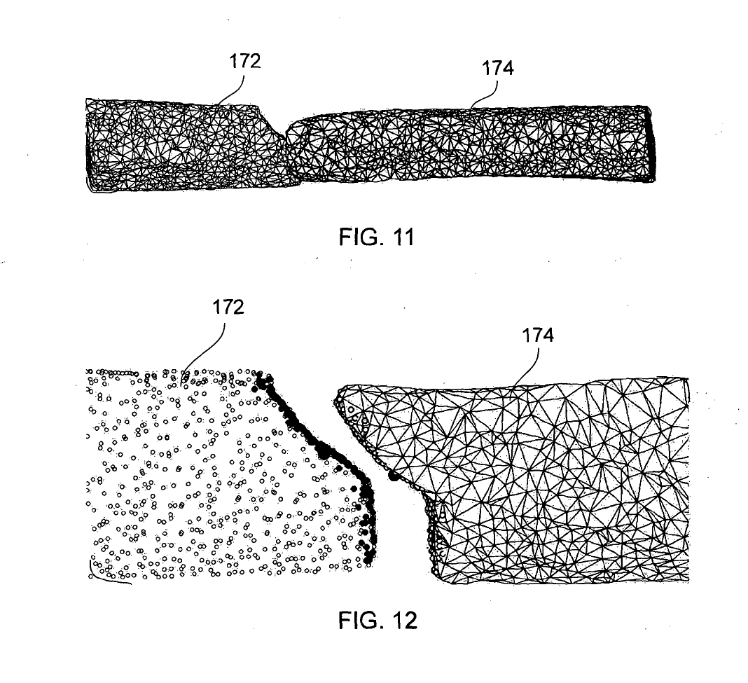

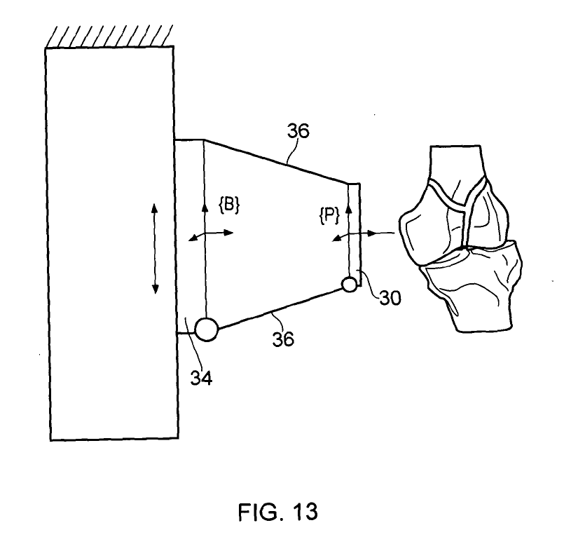

Each point of the point cloud has an x coordinate, a y coordinate and a z coordinate, and these coordinates are stored in a matrix having 3 columns (x, y, z coordinates) and n rows, where a is the number of points in the point cloud. The processing system performs a triangulation on this point cloud to generate a fragment surface layer, as shown in FIG. 11, which is representative of the surfaces of the bone fragments.

The processing system calculates the axis and fracture surfaces of the bone fragments identified in the point cloud. In a first step each bone fragment 172, 174 is defined in two sections (referred to as section A and section B), and the point centre cᵢ (not shown) of each of the sections A and B of each bone fragment is calculated.

The processing system calculates the point centre cᵢ of each section A and B of each bone fragment by calculating the mean position of the x, y and z coordinates of the n points in the point cloud for the selected section. The position of the point centre cᵢ of a section of a bone fragment is at the mean x, y and z coordinates of the selected section of the bone fragment.

Vector algebra is then used by the processing system to calculate an axis vector for each bone fragment 172, 174 by subtracting the coordinates of the point centre of one section of the bone fragment 172, 174 from the point centre of the other section of the bone fragment 172, 174, as indicated in the following Matlab code:

axis_vA=c2−c1 %axis vector 1st fragment
axis_vB=c2−c1 %axis vector 2nd fragment

These axis vectors (shown in FIG. 10) are used in a later step to align the bone fragments specifying four degrees of freedom, namely along two Cartesian axes X and Y and around two Cartesian axes Θₓ and Θ_{y}. This permits lateral and rotational alignment of the bone fragments 172, 174.

The shaft axis of each bone fragment 172, 174 is used by the processing system to extract (i.e. calculate) and plot fracture surfaces of the bone fragment 172, 174. This extraction of the fracture surface is performed based on the assumption that all undamaged surface unit vectors are perpendicular to a unit vector of the calculated axes of the two bone fragments 172, 174. An example Matlab implementation of an algorithm used by the processing system to extract and plot the fracture surface is presented below:

| | |
|---|---|
| v1=tnorm1A; | %array containing unit vectors of each triangulated |
| | surface element (1st fragment) |
| v2=axis_v_nA; | %unit vector of axis vector 1st fragment |

```
%calculate angles between unit axis vector and each surface unit vector
for i=1:length(v1)
    angle(i) = atan2(norm(cross(v1(i,:),v2)),dot(v1(i,:),v2));
    angle(i)=angle(i)*180/pi;
end
%apply constraints to extract fracture surface points for fragment top
and bottom section
for i=1:length(tnorm1A)
    if angle(i)<=70 %&& angle(i)>=100
        plot3(P0A(i,1),P0A(i,2),P0A(i,3),‘x’);
          if P0A(i,3)>110
            ext_stA(i,:)=[P0A(i,1),P0A(i,2),P0A(i,3)];%extracted surf.
Pts 1st Fragment (top)
          elseif P0A(i,3)<80
            ext_sbA(i,:)=[P0A(i,1),P0A(i,2),P0A(i,3)];%extracted surf.
Pts 1st fragment (bottom)
          end
    end
end
```

The algorithm starts by calculating the angle between the unit axis vector and each surface unit vector. Having calculated this angle it can be used as an evaluation criteria to extract surface points for the fragment top (section A) and bottom section (section B) by using two if-functions and by specifying numerical values in order to separate the fragment top and bottom section. The extraction of the surface points is performed under the assumption that all undamaged surface unit vectors are perpendicular to a unit axis vector. If this is not true, surface points must also be points within the fractured surface region.

Having extracted physical quantities such as the axis vector and the fracture surface points of the two fragments the processing system can calculate the rotations and translations required to reduce (i.e. reconstruct) the fractured bone. Typically, such as in the example of a long bone tibia fracture illustrated in FIGS. 7 to 12, this is performed in three steps, namely the registration or alignment of the shaft axes of the fragments, registration of the shaft axis rotation (i.e. a rotation about the axis of a fragment to required for correct alignment of the fracture surfaces) and registration of the distance between the fragments (i.e. a translation required to minimise the distance between the fracture surfaces).

In order to register (i.e. reduce or realign) bone fragments based on their shaft axes a coordinate frame is generated and associated with each fragment 172, 174 by the processing system. The coordinate frame for the first fragment 172 is defined by first selecting the calculated axis vector of the fragment 172 as one of the axes of the coordinate frame. In this example, the axis calculated for the fragment 172 is selected as the X axis of the coordinate frame for that fragment. The same steps are performed for the second fragment 174.

The cross-product of the defined X axes of the fragments 172, 174 gives the Z axis of the coordinate frame, and the cross product of the X axis of the fragment 172, 174 for which the coordinate frame is defined and the Z axis determines the Y axis of the coordinate frame.

Exemplary Matlab code for defining a coordinate frame for fragments of a diapyhseal fracture is presented below:

| | |
|---|---|
| A=axis_v_nA; | %unit axis vector Fragment A (X-axis) |
| B=axis_vn_B; | %unit axis vector Fragment B (X-axis) |
| Z=cross(A,B); | calculate cross product of A and B to obtain Z axis |
| | vector of frame |
| LL=norm(Z); | %Calculate magnitude of Z axis vector |
| Z_n=Z/LL; | %Calculate unit axes vector of axis vector Z |
| % calc frame axis Y | |
| Cy=cross(Z_n,B); | %calculate cross product of unit axis vector Z and |
| | unit axis vector B to obtain Y axis |
| LLL=norm(Cy); | %Calculate magnitude of Y axis vector |
| Y_n=Cy/LLL; | %Calculate unit axes vector of axis vector Y |
| FB=[B Y_n′ Z_n′]; | %assembly 3×3 Coordinate Frame Matrix using |
| | unit axis vectors of X, Y and Z axes |

The coordinate frame for each bone fragment 172, 174 specifies the orientation of the fragment 172, 174 relative to a global body coordinate system (BCS) defined by the matrix

$$BCS = [ \begin{matrix}1 & 0 & 0 \\ 0 & 1 & 0 \\ 0 & 0 & 1\end{matrix} ]$$

All of the surface points (from the point cloud) of each bone fragment 172, 174 are referenced with respect to the BCS frame matrix and therefore represent position vectors with respect to the BCS frame matrix (^{BCS}pBᵢ is the position vector for the fragment 174, ^{BCS}pAᵢ is the position vector for the fragment 172). This allows the position vectors ^{BCS}pBᵢ and ^{BCS}pAᵢ of the surface points of each fragment 172, 174 to be rotated so that they are defined with respect to the coordinate frame matrix FA or FB of that fragment 172, 174 rather than the global BCS frame matrix. This relationship is shown in the formula below applying the inverse of the defined coordinate frame matrix to rotate the vectors ^{BCS}pBᵢ and ^{BCS}pAᵢ.

$${}_{}^{FB}{pB}{}_{i}^{} = {[ {}_{BCS}^{FB} FB ]}^{- 1} \cdot {}_{}^{BCS}{pB}{}_{i}^{}$$

The resulting axial alignment is plotted, as shown in FIG. 11. Exemplary Matlab code to implement this formula is shown below:

```
%% Rotate in Fragment A space
for i=1:length(pA_FA)
    pA_FAr=inv(RRA)*pA_FA(i,:)′;
    pA_FAr2(i,:)=pA_FAr′;
end
plot3(pA_FAr2(:,1),pA_FAr2(:,2),pA_FAr2(:,3),‘ro’), hold on
```

Once the matrix transformation required to achieve the desired axial alignment of the bone fragments 172, 174 has been calculated, the processing system calculates the rotational alignment of the bone fragments 172, 174 around the Cartesian X-axis.

To do this the processing system calculates a reference two-dimensional polyline approximating a two dimensional fracture surface plotted perpendicular to the calculated axis vector X of a selected one of the bone fragments 172, 174, which is used as a reference. In the present example the first fragment 172 is used as the reference fragment. A plurality of further two-dimensional polylines are calculated, approximating a two dimensional surface of the other fragment (in this example the second fragment 174). Each of the plurality of further polylines has a rotational offset, which increases for each successive polyline in small increments between 0 and 360 degrees. By doing this, the optimum rotational axis alignment between the fragments 172 and 174 can be determined by comparing cross correlation coefficients C_{A} of the reference polyline of the first fragment 172 with a cross correlation coefficient C_{Bi} for the second fragment 174 for each rotational increment of the polyline.

The processing system then selects the angle of rotation for which the correlation coefficient C_{Bi} is closest to the cross correlation coefficient C_{A} (i.e. for which the difference value is smallest) as the correct angle of rotation for angular alignment of the bone fragment 174, 172 with respect to the reference bone fragment 172. FIG. 12 illustrates correctly angularly aligned bone fragments 172, 174.

Once the angular rotation required for correct angular alignment of the bone fragments 172, 174 has been calculated, the processing system must calculate the translational movement between the fragments 172, 174 required for correct reduction of the fracture. The translational distance between the fragments 172, 174 can be minimised using the Iterative Closest Point algorithm developed by Besl and McKay (1992).

The translations and rotations of a bone fragment calculated by the processing system for correct reduction of a fracture must be facilitated by the second manipulator 14 and if appropriate the third manipulator 16. The translations and rotations are calculated in terms of a Cartesian coordinate system (operational space or Cartesian space), whereas the manipulators 14, 16 operate on a joint coordinate system (joint space) defined by the coordinates of the joints of the manipulators 14, 16. Therefore, a transformation between the operational space and the manipulator joint space is required, to enable the manipulators 14, 16 to effect the translations and rotations in the operational space calculated by the processing system, and to enable the processing system correctly to determine the joint coordinates of the manipulators 14, 16 based Cartesian inputs (X,Y,Z, θₓ, θ_{y}, θ_{z},) received from the processing system.

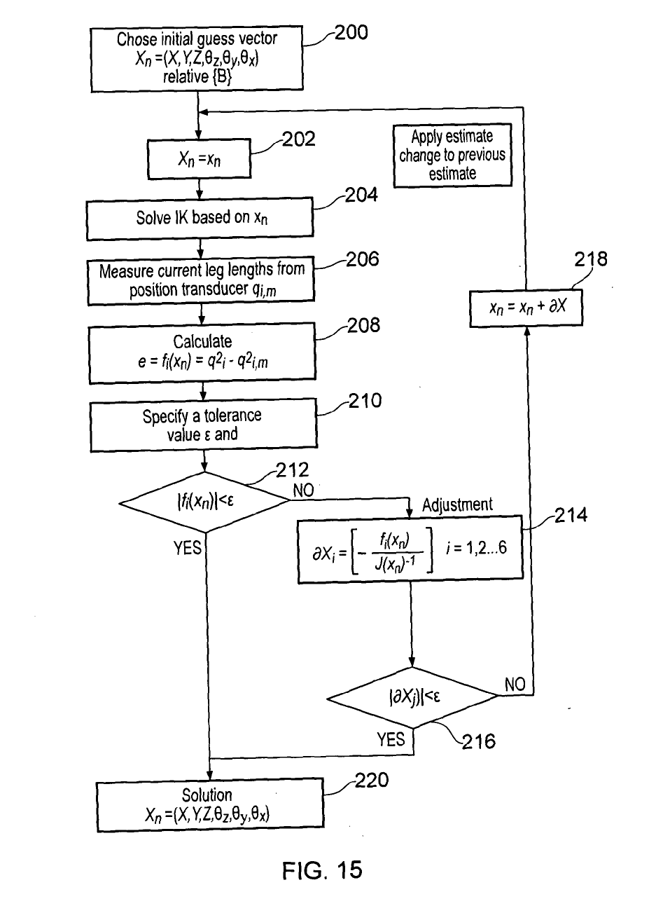

The transformation between Cartesian coordinates and the joint coordinates is referred to as Inverse Kinematics, whilst the reverse transformation, between joint coordinates and Cartesian coordinates, is referred to as Forward Kinematics. An iterative algorithm to solve the Forward Kinematics is described in FIG. 15, whilst a process for the calculation of the Inverse Kinematics required to implement the translations and rotations required for reduction of a fracture is described below.

The entire structure of the second manipulator 14 is fully specified by four design parameters, namely the joint circle diameter dₚ of the moveable platform 30, the joint circle diameter d_{b} of the fixed base 34, the angular joint spacing of the base Θ_{b} and the platform Θₚ, assuming a standard 60 degree offset angle between the base and the platform.

The coordinate frame {P} of the platform 30 relative to the base 34, defined by a coordinate frame {B}, is defined by a position vector linking the origin of {B} and {P}.

Having defined the kinematic model of the manipulators by the four design parameters mentioned above, in a further pre-processing step the processing system calculates angular joint positions of a platform joint Pᵢ (i.e. the joint linking the ith linear actuator 36 to the platform 30) and of a base joint Bᵢ (i.e. the joint linking the ith linear actuator 36 to the to the base 36) as a function of the angular joint spacing Θ_{b} and Θₚ. From the angular joint positions Pᵢ, Bᵢ, joint vectors pᵢ and bᵢ relative to the origin of {P} and {B} can be calculated. A rotation matrix _{B}^{P}R is calculated based on Cartesian angular inputs Θ_{z}, Θ_{y}, Θₓ.

From these initial calculations the processing system can calculate the magnitude of vectors lᵢ (i.e. the magnitude of the vector l for the ith linear actuator 36 of the manipulator), which corresponds to the length of the ith linear actuator 36 using the loop closure equation |lᵢ|=^{B}r+_{B}^{P}R·^{P}pᵢ−^{B}pᵢ, where ^{P}p and ^{B}b are vectors describing the geometry of the mechanism. The vector ^{B}r is a position vector linking origin {B} and {P} and thereby specifying the translational movement of the platform with respect to the fixed base.

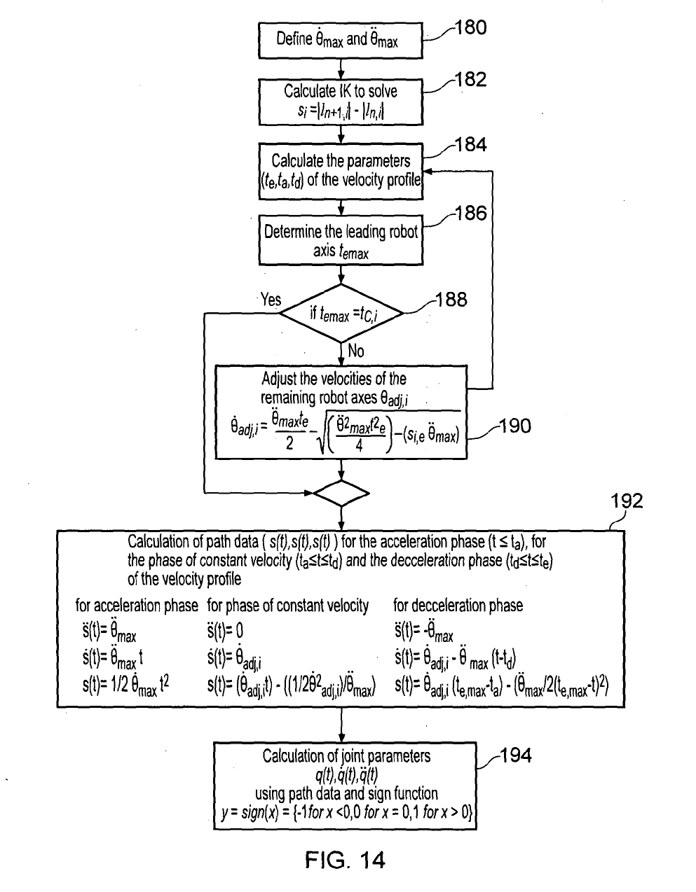

Having calculated the length of each of the linear actuators 36 for the position input the processing system calculates the joint reference parameters (position, speed and acceleration) for every sampling interval q, \dot{q}, \ddot{q} and each linear actuator 36. This is shown in the flow diagram of FIG. 14. These reference data are the transferred to the controller to drive each actuator 36 of the manipulator 14 and to minimize the error between reference data and position feedback received from an absolute displacement sensor associated with each actuator 36 of the manipulator 14.

In a first step 180, maximum velocity \dot{Θ}ₘₐₓ and acceleration {dot over (\ddot{Θ}ₘₐₓ for each of the linear actuators 36 are defined by the operator of the system 10.

At step 182 the processing system solves the loop closure equation (Inverse Kinematics equation) |lᵢ|=^{B}r=_{B}^{P}R·^{P}pᵢ−^{B}pᵢ to obtain the relative position change sᵢ for the ith actuator (where i=1 . . . 6) between the initial magnitude of the leg vector |l_{n,i}| and the new magnitude of the leg vector |l_{n+1,i}| (representing the new length of the actuator 36).

In a further step 184 the processing system calculates parameters tₑ, tₐ, t_{d} of a trapezoidal velocity profile for the change in the length of each linear actuator 36. The change of length occurs in three phases: an acceleration phase, which takes place during time a period from a motion start time t to a time tₐ, a deceleration phase, which takes place from a time t_{d} to a motion end time tₑ, and a constant velocity phase, which takes place during a time period from tₐ to t_{d}.

At step 186 the processing system determines the actuator which will have the longest operational travel time tₑₘₐₓ to perform the positional change based on the parameters specified in steps 180 and 182. The actuator with the longest operational travel time becomes the leading robot axis. At step 188 the processing system compares this maximum travel time tₑₘₐₓ to the calculated travel time end time t_{e,i} for the ith linear actuator 36. If tₑₘₐₓ is not equal to tₑ the joint velocities of the remaining linear actuators 36 of the manipulator are adjusted, in step 190 so that all of the actuators 36 start and end their motion at the same time, using the equation

$${\dot{Θ}}_{i , adj} = \frac{{\ddot{Θ}}_{max} {t}_{e}}{2} - \sqrt{\frac{{\ddot{Θ}}_{max}^{2} {t}_{e}^{2}}{4} - ( {s}_{i , e} {\ddot{Θ}}_{max} )}$$

Once this adjustment has been made, or if tₑₘₐₓ is equal to tₑ, processing passes to step 192, in which path data s(t), \dot{s}(t) and \ddot{s}(t) (i.e. displacement as a function of time, linear velocity as a function of time and linear acceleration as a function of time) for the acceleration phase, the constant velocity phase and the deceleration phase are calculated using the following equations:

For the acceleration phase

\ddot{s}(t)=\ddot{Θ}ₘₐₓ

\dot{s}(t)=\ddot{Θ}ₘₐₓt

s(t)=1/2\ddot{Θ}ₘₐₓt²

For the phase of constant velocity

$$\ddot{s} ( t ) = 0$$

$$\dot{s} ( t ) = {\dot{Θ}}_{adj , j}$$

$$s ( t ) = ( {\dot{Θ}}_{adj , i} t ) - ( \frac{( \frac{1}{2} {\dot{Θ}}_{adj , i}^{2} )}{{\ddot{Θ}}_{max}} )$$

For the deceleration phase

$$\ddot{s} ( t ) = - {\ddot{Θ}}_{max}$$

$$\dot{s} ( t ) = {\dot{Θ}}_{adj , i} - {\ddot{Θ}}_{max} ( t - {t}_{d} )$$

$$s ( t ) = {\dot{Θ}}_{adj , i} ( {t}_{e max} - {t}_{a} ) - ( \frac{{\ddot{Θ}}_{max}}{2 {( {t}_{e max} - t )}^{2}} )$$

Having calculated the path data, processing moves to step 194, in which the processing system calculates joint parameters q(t), \dot{q}(t) and \ddot{q}(t) using the path data and the sign function y=sign(x)={−1 for x<0, 0 for x=0, 1 for x>1} to verify the joint parameters and to distinguish between positive and negative motions. With the parameters (extension s, maximum speed and maximum acceleration) specified for the linear actuators 36 and the joint parameters calculated the processing system is able to cause the required synchronic movement of the linear actuators 36 to effect the desired manipulation of the bone fragment.

To calculate the Forward Kinematics required to transform joint coordinates reported by the manipulators 14 into Cartesian coordinates an iterative algorithm is used. FIG. 15 is a flow chart illustrating steps taken by the processing system in calculating the Forward Kinematics.

In a first step 200, an initial guess vector Xₙ relative to the base 34 {B}, containing Cartesian coordinates X, Y, Z and angles Θ_{z,} Θ_{y,} Θₓ, is selected.

In steps 202 and 204 a first estimate X₀ is set to equal the initial guess vector x and the Inverse Kinematics are solved based on x using the equation |lᵢ|=^{B}r+_{B}^{P}R·^{P}pᵢ−^{B}pᵢ set out above to calculate qᵢ qᵢ=|lᵢ|. The current lengths of the linear actuators 36 termed in this algorithm measured by an absolute displacement transducer in step 206, are compared to the calculated lengths (using the Inverse Kineamatics) of the linear actuator qᵢ and an error is obtained in step 208.

Subtracting q_{i,m} from qᵢ and defining the Forward Kinematics problem as ‘root finding problem’, an error in mm is obtained as mentioned above and compared in step 212 to a suitable and specified tolerance value ε in step 210. To find the roots of the error function defined in 208 the Newton-Raphson method is used as a standard algorithm to solve nonlinear equations numerically. Single steps to perform this method are briefly outlined for the present case in 212, 214, 216 and 216.

If |fᵢ(xₙ)|<ε is true, i.e. the difference between the calculated length qᵢ and measured actuator length q_{i,m} is smaller than the selected tolerance value ε, a solution for the Forward Kinematics has been found. In this position the calculated location of the robot platform defined by the Cartesian vector Xᵢ (where i donates a single iteration step) corresponds to the measured actuator length q_{i,m} taking the specified tolerance value e into account.

In case |fᵢ(xₙ)| is larger than ε, the Cartesian coordinates summarized by X₀=xₙ are adjusted using the term

$$[ - \frac{{f}_{i} ( {x}_{n} )}{{J ( {x}_{n} )}^{- 1}} ]$$

specified in step 214 where J represents the Inverse Jacobian Matrix.

If the absolute value of the adjustment δXᵢ is less than the specified tolerance value ε the correct Cartesian position of the platform 30 in space X has been found based on the joint space input. If not, the calculated adjustment δX in step 214 is added to the initial guess vector xₙ at step 218 and the steps 202-220 are repeated until the correct vector X describing the position and orientation of the platform 30 in Cartesian coordinates is found.

In the embodiment of the system 10 described above with reference to FIG. 5, features of the reference bone and bone fragments of a fracture are identified either automatically or by a qualified surgeon based on a graphical representation of the fracture based on the medical images (scans) taken of the fracture prior to and during the surgical procedure for reducing the fracture, and the processing system calculates, from the identified features, manipulations of the reference bone and bone fragments required to effect the desired reduction, and motion reference signals for the manipulations for the controller to cause the first, second and third manipulators 12, 14, 16 to effect the calculated manipulations.

In an alternative second embodiment of the system, the processing system presents a graphical user interface that enables a surgeon to perform a virtual manual pre-reduction on a graphical representation of the fracture. The processing system records manipulations of the virtual bone fragments made by the surgeon, and from these recorded manipulations calculates the motion reference signals required to cause the first, second and third manipulators 12, 14, 16 to effect the manipulations performed on the virtual fragments.

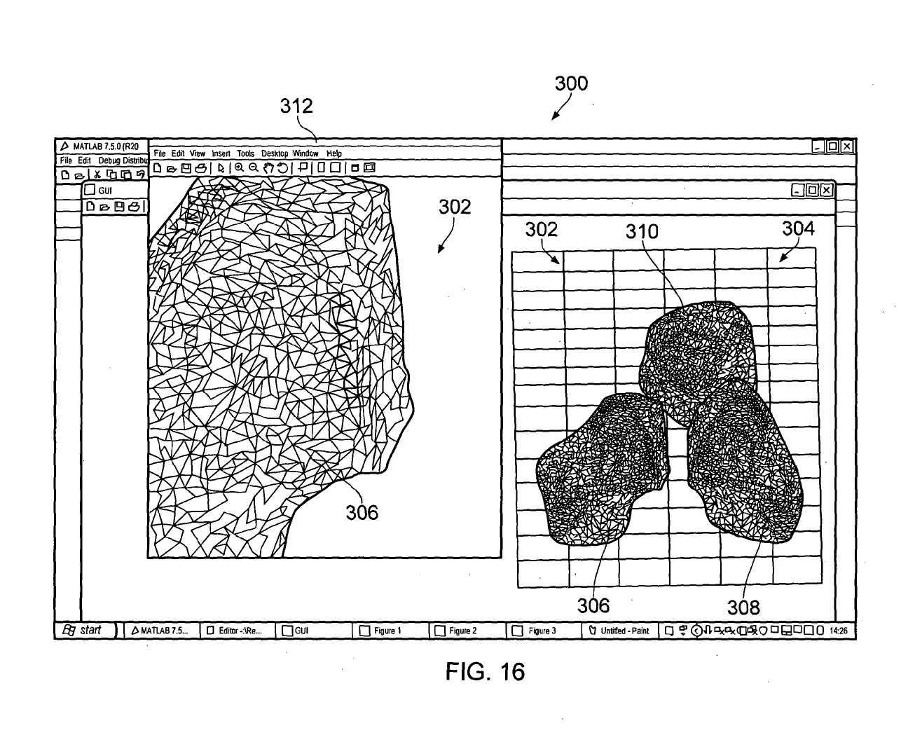

FIG. 16 is a screenshot showing a graphical user interface 300 employed in this alternative embodiment, providing a graphical representation of a bone fracture prior to the virtual pre-reduction discussed above.

In the second embodiment to which FIG. 16 relates, the steps of segmentation and generation of fragment surface described above in relation to the first embodiment are performed to generate graphical representations 302 of the bone fragments of the fracture which are displayed by the processing system. The image 304 in the right-hand window of the screenshot of FIG. 16 shows a graphical representation of the three bone fragments 306, 308, 310 of the fracture case, whilst the left-hand window 312 shows an enlarged view of the fragment 306, illustrating more clearly the calculated fracture surface.

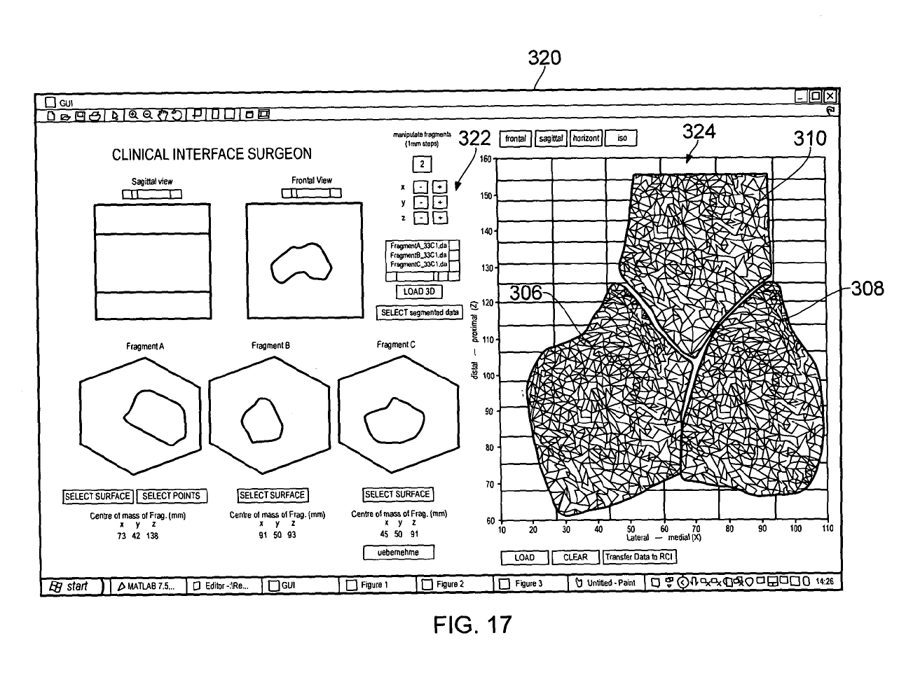

FIG. 17 is a screenshot showing a further graphical user interface 320 employed in the second embodiment to permit manipulation of the graphical representations of the bone fragments 306, 308, 310, to achieve a simulated virtual pre-reduction of the fracture. The graphical user interface 320 provides a “virtual joystick” 322 by means of which the surgeon is able to manipulate (translate or rotate) a selected representation of a fragment 306, 308, 310. The effect of such manipulation is shown on a graphical representation 324 of the fracture, allowing the surgeon to see the effect of the manipulations. In this way, the surgeon is able to simulate the manipulations required to each fragment 306, 308, 310 before any manipulation of the actual bone fragments takes place to determine precisely the manipulations required for optimum reduction of the fracture.

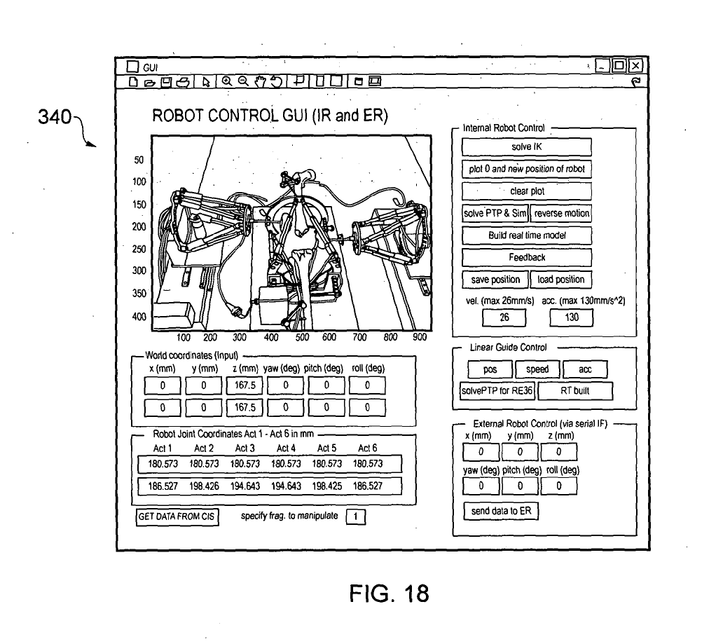

The processing system records the manipulations performed on the virtual bone fragments 306, 308, 310, and calculates, from the recorded manipulations, the reference signals which are required to effect the desired manipulations of the actual bone fragments by the first, second and third manipulators 12, 14, 16. These reference signals are transferred to a further graphical user interface, shown at 340 in FIG. 18, by means of which the first, second and third manipulators 12, 14, 16 are controlled.

It will be appreciated that the processing and calculation steps will typically be performed by a software program being executed by processing system such as a general purpose computer.

Accordingly, the present invention extends to a computer program which, when executed by appropriate processing means, performs processing steps as described above.

1. A system for reduction of bone fractures, the system comprising:
a first manipulator for manipulating a bone section of the fracture, the first manipulator being attachable to the bone section by means of a percutaneous attachment device;
a second manipulator for manipulating a first bone fragment of the fracture, the second manipulator being attachable to the first fragment by means of a percutaneous attachment device;
a processing system configured to determine reference signals for the first and second manipulators required to effect manipulations of the bone section and the first fragment required for correct anatomical reduction of the fracture; and
a controller configured to control the first and second manipulators to cause them to perform the manipulations of the bone section and the first bone fragment.

2. The system according to clam 1 wherein the first manipulator is a parallel manipulator comprising first and second end sections connected by a plurality of linear actuators.

3. The system according to claim 1 further comprising a third manipulator for manipulating a second bone fragment of the fracture, the third manipulator being attachable to the fragment by means of a percutaneous attachment device, wherein the processing system is configured to determine reference signals for the third manipulator required to effect manipulations of the second fragment required for correct anatomical reduction of the fracture and the controller is further configured to control the third manipulator to perform the manipulations of the second bone fragment.

4. The system according to claim 1 further comprising a fourth manipulator having a tool for removing fragments of bone that cannot be manipulated.

5. The system according to claim 1 wherein the second and/or third and/or fourth manipulator is a parallel manipulator comprising a fixed base and a moveable platform connected to the fixed base by a plurality of linear actuators.

6. The system according to claim 5 wherein the fixed base is connected to the moveable platform by six linear actuators.

7. The system according to claim 1 wherein the controller is configured to cause the manipulators to perform the manipulations of the reference bone and the bone fragment(s) substantially simultaneously.

8. The system according to claim 1 wherein the processing system is configured to:
receive an image of the fracture;
segment the image of the fracture to identify fracture surfaces of the reference bone and the bone fragment(s) of the fracture;
generate a fragment surface layer for the reference bone and the fragment(s) representative of surfaces of the reference bone and the bone fragments;
display a graphical representation of the reference bone and the bone fragments; receive a user input;
manipulate the graphical representation to simulate reduction of the fracture based on the user input received;
record the manipulations of the graphical representation; and,
based on the recorded manipulations, determine the reference signals for the manipulations required for correct anatomical reduction of the fractured bone.

9. The system according to claim 8 wherein the user input is received by means of a virtual joystick presented as part of a graphical user interface by the processing system.

10. A method for calculating manipulations required to effect an anatomical reduction of a bone fracture, the method comprising the steps of:
segmenting an image of the fracture to identify fracture surfaces of bone fragments of the fracture;
generating a fragment surface layer for each fragment representative of surfaces of the bone fragment;
calculating an axis of each bone fragment;
calculating fracture surfaces for each bone fragment; and
calculating, based on the axes and fracture surfaces calculated, manipulations required for reduction of the fracture.

11. The method according to claim 10 wherein the segmenting of the image of the fracture comprises calculating Hounsfield intensity values for structures shown in the image and identifying the fracture surfaces from the Hounsfield intensity values.

12. The method according to claim 10 wherein generating the fragment surface layer for each fragment comprises using data from the segmented image to generate a point cloud representing each bone fragment and performing a triangulation on the point cloud.

13. The method according to any one of claims 10 wherein calculating the axis of a bone fragment comprises defining two sections of the bone fragment, calculating the point center of each of the defined sections of the fragment and calculating an axis vector for the bone fragment by subtracting the point center of one section from the point center of the other section.

14. The method according to claim 13 wherein calculating the point center of a section of the bone fragment comprises calculating the mean x, y and z coordinates for each point of the point cloud in the section.

15. The method according to any one of claims 10 wherein calculating the manipulations required for reduction of the fracture comprises calculating a manipulation required for axial alignment of the fragments, calculating an angle of rotation required for fracture surface alignment and calculating a translation required to close a gap between the bone fragments.

16. The method according to claim 15 wherein calculating the manipulation required for axial alignment of the fragments comprises calculating a transformation required to align the axis vectors of the fragments.

17. The method according to claim 15 wherein calculating the angle of rotation required for fracture surface alignment comprises:
generating a first polyline representative of the fracture surface of a first, reference fragment;
generating a plurality of second polylines representative of the fracture surface of a second fragment, each second polyline having an incremental angular offset with respect to the first polyline; and
performing a comparison of the first and each of the plurality of second polylines, wherein the angle of rotation required is determined by calculating the angular offset for which the comparison determines that the first and second polylines are most similar.

18. The method according to claim 17 wherein performing the comparison comprises:
calculating a first cross correlation coefficient for the first polyline;
for each of the plurality of second polylines, calculating a second cross correlation coefficient; and
calculating a difference value between the first and second cross correlation coefficients, wherein the angle of rotation required is the angular offset of the second polyline for which the difference value is smallest.

19. A method for calculating manipulations required to effect an anatomical reduction of a bone fracture, the method comprising the steps of:
segmenting an image of the fracture to identify fracture surfaces of bone fragments of the fracture;
generating a fragment surface layer for each fragment representative of surfaces of the bone fragment;
displaying a graphical representation of the bone fragments;
receiving a user input;
manipulating the graphical representation of one of the bone fragments to simulate reduction of the fracture based on the user input received;
recording the manipulations of the graphical representation of the bone fragment; and,
based on the recorded manipulations, determining reference signals for manipulations of the fragments of fractured bone required for correct anatomical reduction of the fractured bone.

20. The method according to claim 19 wherein the user input is received by means of a virtual joystick presented as part of a graphical user interface.

21. The system according to claim 1 wherein the processing system is configured to perform the method of:
segmenting an image of the fracture to identify fracture surfaces of bone fragments of the fracture;
generating a fragment surface layer for each fragment representative of surfaces of the bone fragment;
calculating an axis of each bone fragment;
calculating fracture surfaces for each bone fragment; and
calculating, based on the axes and fracture surfaces calculated, manipulations required for reduction of the fracture;
and the controller is configured to cause the manipulators to perform the calculated manipulations.

22. A computer program which, when executed on an appropriate processing system, performs the method of claim 10.
